(19) United States
(12) Patent Application Publication
(10) Pub. No.: US 2016/0136602 A1
(43) Pub. Date: May 19, 2016

(54) COLLECTOR PIPE FOR A RADIAL REACTOR COMPRISING SOLID FILLETS

(71) Applicant: IFP ENERGIES NOUVELLES, Rueil-Malmaison Cedex (FR)

(72) Inventors: Frederic BAZER-BACHI, Irigny (FR); Cecile PLAIS, Les Haies (FR); Mehdi LE MOEL, Oullins (FR); Eric SANCHEZ, Saint Genis Laval (FR)

(73) Assignee: IFP ENERGIES NOUVELLES, Rueil-Malmaison Cedex (FR)

(21) Appl. No.: 14/940,399

(22) Filed: Nov. 13, 2015

(30) Foreign Application Priority Data
Nov. 14, 2014 (FR) 1461003

(51) Int. Cl. B01J 8/00; B01D 46/24; C07C 5/27; B01J 8/08; C10G 11/00; C07C 6/04

(52) U.S. Cl. CPC B01J 8/006; C10G 11/00; C07C 6/04; C07C 5/27; B01J 8/085; B01D 46/2403; B01J 2208/00752; B01J 2208/00761; B01J 2208/00884; B01J 2208/00893

(57) ABSTRACT

This invention relates to a collector pipe (8) that is permeable to a gaseous fluid and impermeable to catalyst particles, with an approximately cylindrical shape extending along an approximately vertical axis having a first surface that can be in contact with the catalyst particles and a second surface opposite the first surface, the pipe containing a plurality of shaped vertical wires (11) having a first face (12) that can be in contact with the catalyst particles and a second face (13) opposite the first face (12), the shaped wires (11) being integral with a plurality of horizontal support rings (14) by their second face (13), the shaped wires (11) and the support rings (14) defining a plurality of openings.

This invention relates to a collector pipe for moving-bed units having a radial circulation of the feedstock and of the reagents from the periphery of the reaction chamber toward the center. A person skilled in the art characterizes as “radial” a flow of gaseous reagents taking place through a generally mobile catalytic bed according to a set of directions corresponding to radii that are oriented from the periphery toward the center, or from the center of the chamber toward the periphery. This invention also has as its object a reactor having a radial bed comprising a pipe for collecting reaction effluent according to the invention. Finally, the invention relates to a method for catalytic conversion of a feedstock of hydrocarbons using a radial-bed reactor.

### STATE OF THE ART

The most representative unit of this type of radial flow is the regenerative reforming of hydrocarbon fractions of the gasoline type that can be defined as having a distillation interval of between 80 and 250° C. The field of application of this invention, however, is broader, and in addition to catalytic reforming of gasolines, there can be cited the skeletal isomerization of various C4, C5 olefinic fractions, or even the metathesis method for producing propylene, for example. This method list is not exhaustive, and this invention can be applied to any type of catalytic method having radial flow and gaseous feedstock. Thus, in the context of new energy technologies, the ethanol to diesel method, for example, could use this type of technology.

Some of these radial-bed units, including regenerative reforming, rely on a flow of the so-called moving-bed catalyst, i.e., a slow gravity flow of the confined catalyst particles (or catalytic bed) in an annular space limited by the wall of the reactor and an interior wall that is permeable to gas and impermeable to the catalyst grains, which corresponds to the collector pipe (or central collector) that recovers the reaction effluents. Alternatively, the catalytic moving bed can be confined in a generally annular space, formed between a so-called “external” grid and the collector pipe that are arranged preferably in a concentric manner. The so-called “external” grid can be formed by an assembly of grid elements in the shape of shells (scallops in English terminology).

The gaseous feedstock is generally introduced through the external periphery of the annular bed and passes through the catalytic bed in a manner that is approximately perpendicular to the vertical direction of flow of the latter. The reaction effluents are then recovered in the collector pipe (or collector).

The use of this type of reactor is, however, limited in terms of feedstock flow. Actually, a feedstock flow that is too high will lead to a phenomenon of jamming of the catalyst against the central collector (or “pinning” according to English terminology). The force exerted by the feedstock circulating radially from the external periphery of the catalyst bed toward the center on the bed of catalyst grains presses them against the wall of the central collector, which increases the frictional stress that then resists the sliding of the grains along the wall. If the feedstock stream is high enough, then the friction force that results from it is enough to bear the weight of the catalytic bed so that the gravity flow of the catalyst grains ceases, at least in certain areas adjacent to the wall of the central collector. In these areas, the catalyst grains are then said to be “jammed” (“pinned” according to English terminology) by the gas flow and are kept immobile against the wall of the collector. The phenomenon of immobilization of the catalyst grains should be strongly avoided in reactors for catalytic reforming of hydrocarbon feedstock to the extent that it promotes the reactions of deactivation of the catalyst (for example by coking), thus preventing the continued exploitation of the reactor. Potentially, when the catalyst cake becomes too thick along the pipe, it is then necessary to reduce the flow of gas to be treated or even to stop the unit for the purpose of unclogging said pipe.

One purpose of the invention is therefore to propose a new type of pipe for collecting gaseous effluent for a radial-bed reactor whose design makes it possible to limit the phenomena of “pinning” of the catalyst grains. Thus, a reactor using the effluent collector according to the invention can be operated for longer periods and/or makes it possible to use larger hydrocarbon feedstock flows (increase of the capacity of the unit).

### SUMMARY OF THE INVENTION

This invention therefore has as its object a collector pipe that is permeable to a gaseous fluid and impermeable to catalyst particles, with an approximately cylindrical shape extending along an approximately vertical axis having a first surface that can be in contact with the catalyst particles and a second surface opposite the first surface, the pipe comprising a plurality of shaped vertical wires having a first face that can be in contact with the catalyst particles and a second face opposite the first face, the shaped wires being integral with a plurality of horizontal support rings by their second face, the shaped wires and the support rings defining a plurality of openings. The pipe further comprises a plurality of solid fillets, impermeable to gaseous fluid and to particles, which are integral with the first face of the shaped wires and/or placed and held between two vertical wires, and the solid fillets form an angle of inclination a in relation to the horizontal of between 10° and 90°.

Surprisingly, the applicant has found that a collector pipe comprising solid fillets in contact with the catalyst particles is less subject to the “pinning” phenomenon for the same feedstock flow compared with a collector pipe according to the prior art.

In the context of the invention, the term “impermeable to particles” is defined as the fact that the openings through which the gaseous fluid diffuses are smaller than the minimum distance taken between two opposite points of the particle so as not to allow said particle to pass through the openings.

The total surface occupied by the solid fillets is preferably between 1 and 30% of the total surface developed by the pipe, in a more preferred way between 2 and 20% of the total surface developed by the pipe.

According to the invention, the fillets extend over a height of between 10 and 100% of the total height of the pipe, preferably between 40 and 100% and in a more preferred way between 50 and 100% of the total height of the pipe.

In one embodiment, the solid fillets are placed in the upper half and/or in a zone located in the lower half of the pipe. Alternatively, the solid fillets are arranged to cover both a portion of the lower and upper halves of the height of the pipe.

According to the invention, the solid fillets can also extend from the upper end and/or from the lower end of the pipe.

In a particular embodiment, the pipe comprises successively and alternately a solid fillet extending in a zone located in the upper half of the pipe and a solid fillet extending in a zone located in the lower half of the pipe.

According to another embodiment, the solid fillets placed in the upper half of the pipe are arranged facing the solid fillets placed in the lower half of the pipe, and the sum of the heights of the solid fillets facing one another is between 20 and 100% of the total height of the pipe.

Preferably, the solid fillets are parallel to one another. Preferably, the solid fillets are uniformly spaced.

In one embodiment, the collector pipe comprises solid fillets forming an angle α in relation to the horizontal that is different by 90° and in which two solid fillets are placed so as to meet by forming a “V” or intersecting by forming an “X.”

The invention also relates to a reactor having radial circulation of gaseous fluid comprising:
an external casing defining a chamber extending along a vertical main axis and containing a reaction zone comprising a bed of catalyst particles;
at least one input means of a feedstock;
at least one output means of the effluent produced by the catalytic reaction;
at least one input means of the catalyst to introduce the catalyst into the reaction zone;
at least one output means of the catalyst coming out from the reaction zone;
a pipe for collecting effluent according to the invention, in communication with the output means of the effluent, placed in the reaction zone, it being understood that the solid fillets are in contact with the catalyst particles of the catalytic bed, and
optionally, a cylindrical grid that is permeable to gases and impermeable to catalyst particles placed in the chamber in a manner that is concentric in relation to the collector pipe.

According to one embodiment of the reactor, the cylindrical grid is placed between the casing and the collector pipe so that the reactor comprises an annular zone for external distribution between the casing and the cylindrical grid, an annular catalytic zone between the cylindrical grid and the collector pipe, and a collector space delimited by the collector pipe and in which the first face of the wires of the collector pipe is in contact with the catalytic bed.

According to another embodiment of the reactor, the collector pipe is placed between the casing and the cylindrical grid so that the reactor comprises an external annular collector zone between the casing and the collector pipe, an annular catalytic zone between the cylindrical grid and the collector pipe, and a collector space delimited by the cylindrical grid and in which the first face of the wires of the collector pipe is in contact with the catalytic bed.

According to one embodiment of the reactor, the cylindrical grid is an assembly of grid elements in the shape of shells extending along the vertical axis in the reactor.

According to one embodiment of the reactor, the cylindrical grid is not present, and the reactor comprises a plurality of tubes for distribution of the gaseous fluid, connected to a distribution box, immersed in the reaction zone.

The invention relates to a method for catalytic conversion of a hydrocarbon feedstock using a reactor according to the invention, in which:
the hydrocarbon feedstock is sent continuously in gaseous form into a catalytic bed contained in the reactor;
the hydrocarbon feedstock passing radially through the catalytic bed is put in contact with the catalyst so as to produce a gaseous effluent; and
said effluent is drawn off after it passes through the collector pipe.

The method according to the invention can use a catalytic moving bed that involves continuously sending and drawing off the catalyst.

The catalytic conversion reaction can be selected from among a catalytic reforming reaction, a skeletal isomerization of olefins, metathesis for the production of propylene, and an oligomeric cracking reaction.

### DETAILED DESCRIPTION OF THE INVENTION

The other characteristics and advantages of the invention will become clear from reading the following description, given solely by way of illustration and nonlimiting, and to which are attached:

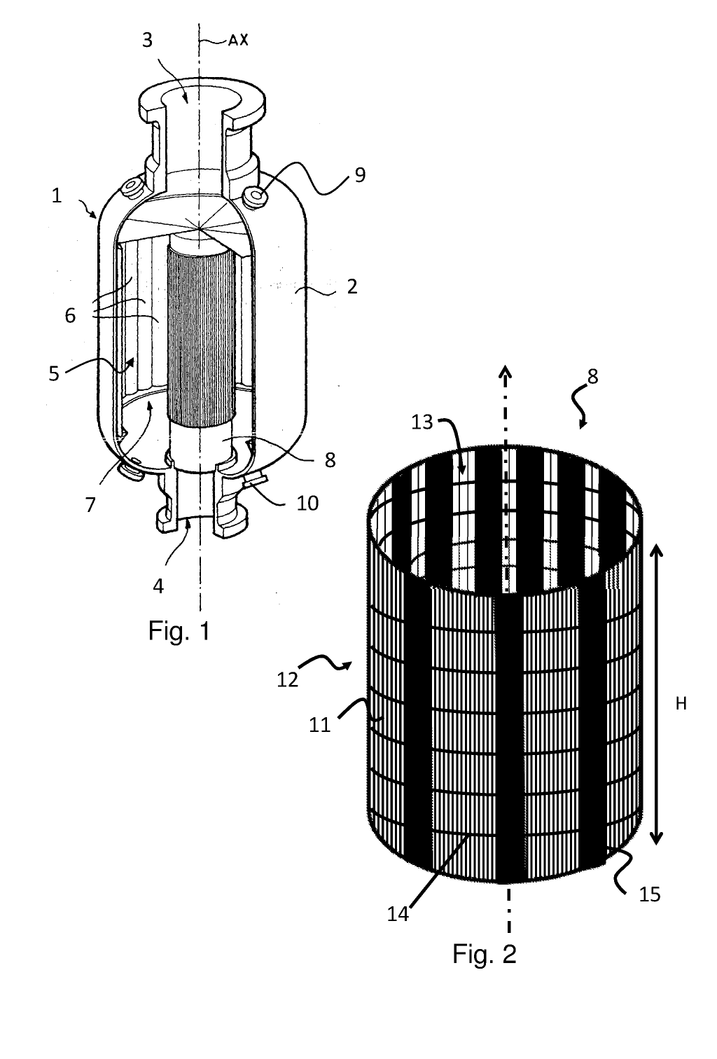

FIG. 1, which shows a perspective view with a partial view of a radial flow reactor according to the prior art;

FIG. 2, which is a perspective view of a pipe according to the invention;

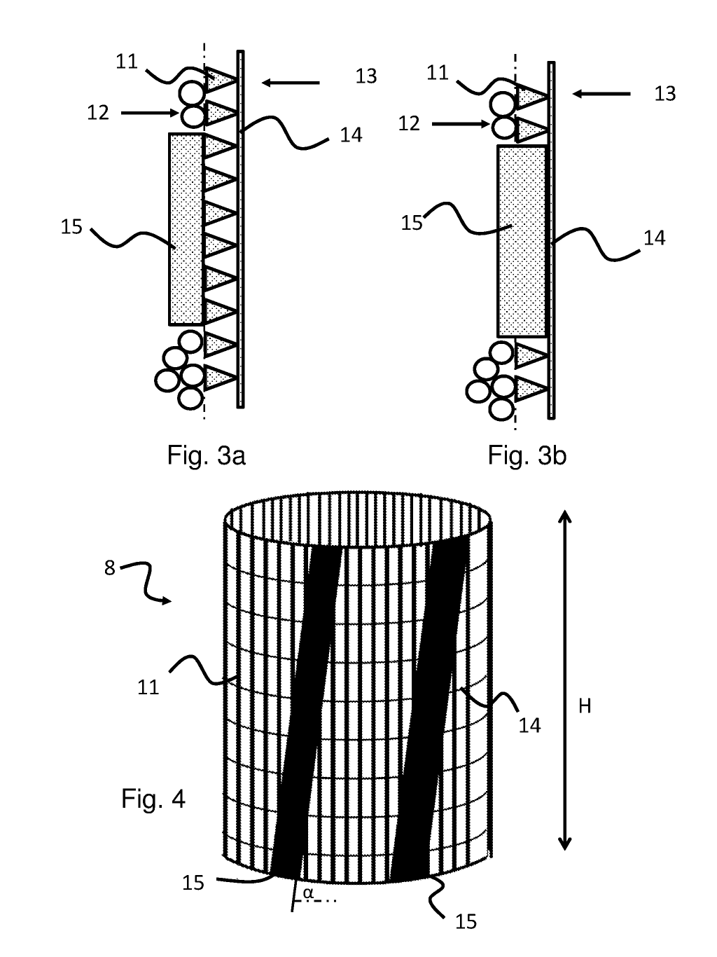

FIGS. 3a and 3b are detailed cutaway views along a plane that is perpendicular to the vertical axis of the pipe of FIG. 2;

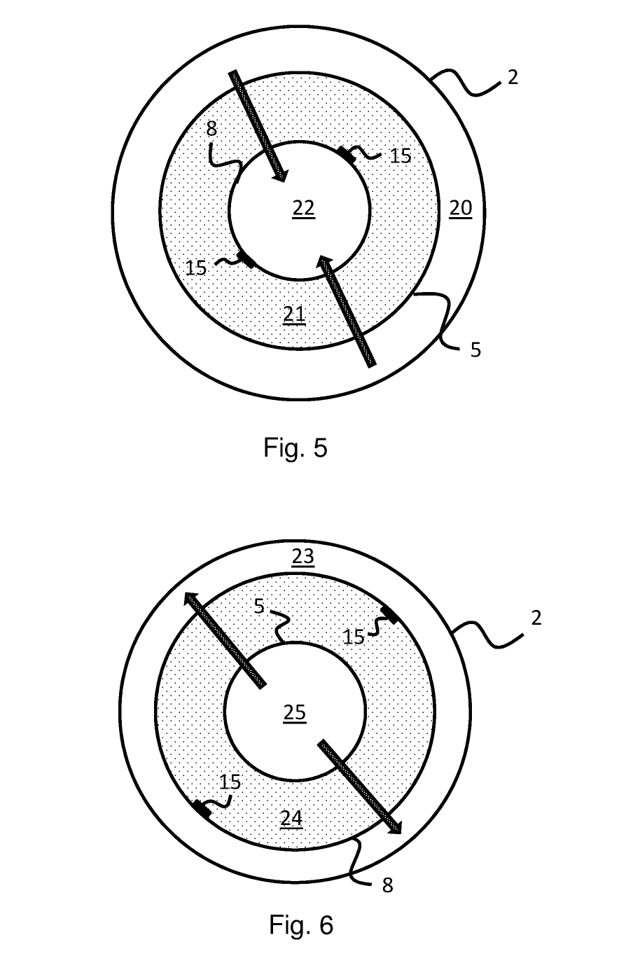

FIG. 4 is a perspective view of a pipe according to the invention according to another embodiment;

FIG. 5 is a cutaway view of a reactor according to the invention along a plane that is perpendicular relative to the main axis of the reactor;

FIG. 6 is a cutaway view of a reactor according to the invention along a plane that is perpendicular relative to the main axis of the reactor;

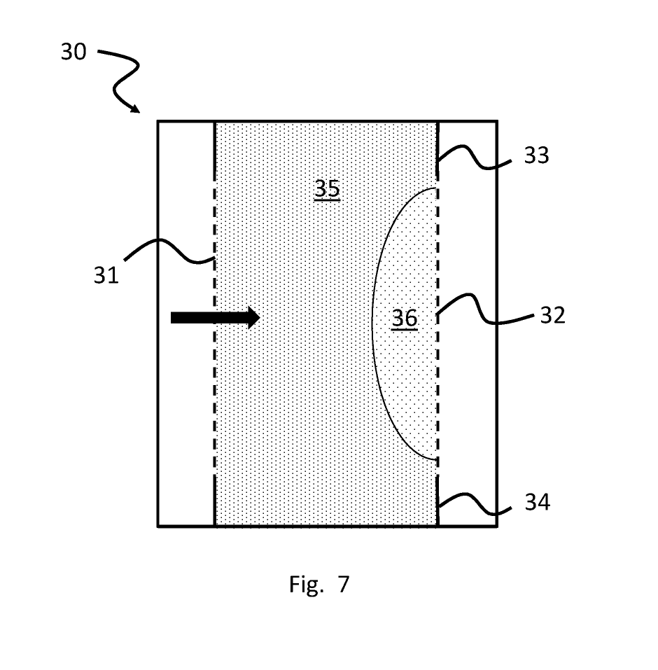

FIG. 7 is a diagrammatic view of a mockup used for studying the pinning.

With reference to FIG. 1, a radial flow reactor 1 according to the prior art appears outwardly in the form of a carboy forming a cylindrical chamber 2 that extends along an axis of symmetry AX. In its upper part, the chamber 2 comprises a first opening 3, and in its lower part, the chamber 2 comprises a second opening 4. The openings 3 and 4 are intended respectively for the input and the output of a fluid passing through the reactor 1.

Inside this cylindrical tank is arranged a catalytic bed 7 having the shape of a vertical cylindrical ring limited on the interior side by a central cylindrical tube 8 formed by a so-called “internal” grid holding the catalyst and on the exterior side by another so-called “external” grid 5 either of the same type as the internal grid or by a device consisting of an assembly of shell-shaped grid elements 6 extending longitudinally, as shown in FIG. 1. These shell-shaped grid elements 6 forming pipes are also known under the English term “scallops.” These pipes 6 are held by the tank and flattened against the internal face of the chamber, parallel to the axis AX, to form an approximately cylindrical internal casing. The shell-shaped grid elements 6 are in direct communication with the first opening 3 via their upper end to receive a gaseous flow of feedstock. The gaseous flow spreads through the perforated wall of the pipes 6 to pass through the bed of catalytic solid particles 7 by converging radially toward the center of the reactor 1. The feedstock is thus put in contact with the catalyst to undergo chemical transformations, for example a catalytic reforming reaction, and to produce an effluent from the reaction. The effluent from the reaction is then collected by the central cylinder tube 8 (or collector pipe) extending along the axis AX and also having a perforated wall. This central cylinder 8 (or collector pipe) here is in communication with the second opening 4 of the reactor via its lower end.

During operation, the gaseous fluid introduced in the first opening 3 radially passes through the “external” grid 5, and then radially passes through the catalyst particle bed 7 where it is put in contact with the catalyst so as to produce an effluent that is eventually collected by the central cylinder 8 and evacuated through the second opening 4.

Such a reactor can also operate with a continuous gravity flow of catalyst in the annular catalytic bed 7. In the case of FIG. 1, the reactor 1 further comprises means for introducing the catalyst 9 into the annular bed, placed in an upper part of the reactor, and means for drawing off the catalyst 10 that are arranged in a lower part of the reactor.

Alternatively, as a means for distribution of the gaseous fluid in a replacement of the external grid, the reactor can use a plurality of fluid distribution tubes, directly immersed in the catalytic bed and the tubes being connected to a distribution box. These distribution tubes, closed at their distal end, for example with a circular cross-section, are formed by a grid or a perforated plate that is permeable to gas and impermeable to catalyst grains.

A first embodiment of a collector pipe according to the invention is now described with reference to FIGS. 2, 3a and 3b.

With reference to FIG. 2, the pipe 8 appears in the form of a vertical cylindrical ring consisting of an assembly of shaped metal wires 11 placed parallel to one another along the vertical axis of the pipe. For example, the shape of the wires can be in a V. As indicated in FIGS. 3a and 3b, the vertical wires have a first face 12 and an opposite second face 13. In the context of the invention, the first face 12 relates to the face that is in contact with the catalyst particles of the catalytic bed when the pipe is used in a radial reactor. The second face that can be designated by the term “back” corresponds to the face that is not in contact with the catalyst bed when the pipe is used in a radial reactor.

The wires are kept integral with one another by a set of horizontal metal support rings 14 that are welded to the second face of the vertical wires at any point of contact with the latter. The support rings 14 are preferably placed uniformly along the height of the pipe. Thanks to this arrangement of the vertical wires 11 and of the support rings 14, the pipe 8 has a perforated wall on its periphery. The placement of the wires and of the rings is such that the openings formed are able to allow a gaseous fluid to spread while retaining the catalyst particles.

According to the invention, the pipe 8 further comprises a plurality of solid fillets 15 that are integral with the vertical wires 11 in the area of their first surface 12 (see FIG. 3a). In the embodiment of FIG. 2, the solid fillets 15 extend parallel to the vertical axis of the pipe, i.e., the angle of inclination a of the solid fillets relative to the horizontal is equal to 90°.

In the context of the invention, the solid fillets 15 are elements that are impermeable to the gaseous fluid and to the particles. For example, the fillets appear in the form of metal plates welded to the vertical wires on their first face 12.

With reference to FIG. 3b, which is another embodiment of the pipe, the solid fillets 15 are inserted between two vertical shaped wires 11 and are integral with and supported directly by the support rings 14.

In the embodiment of FIG. 2, the pipe 8 comprises solid fillets 15 that extend over the entire height H of the pipe 8 and on its periphery. Furthermore, the solid fillets 15 are placed along the vertical axis and in a manner parallel to one another with preferably uniform spacing between the solid fillets.

In the context of the invention, the number of solid fillets as well as their dimensions are determined so that the total surface covered by the fillets is between 1 and 30% of the total surface developed by the pipe, preferably between 2 and 20% of the total surface developed by the pipe.

According to the invention, each solid fillet can extend over a height of between 10 and 100% of the total height of the pipe. Preferably, the fillets extend over a distance of between 50% and 100% of the total height of the pipe.

The arrangement of the solid fillets on the periphery of the pipe can assume different configurations. For example, all of the solid fillets are placed in a zone located in the upper half of the pipe and/or in a zone located in the lower half of the pipe.

Alternatively, the solid fillets cover both a portion of the zones located in the lower and upper halves of the pipe. In another embodiment, the solid fillets are placed successively and alternately in a zone located in the lower half of the pipe and in a zone located in the upper half of the pipe.

According to another placement, the solid fillets arranged in a zone located in the upper half of the pipe are facing solid fillets placed in a zone located in the lower half of the pipe, and the sum of the heights of the solid fillets facing one another is between 20% and 100% of the total height of the pipe.

Another embodiment of the pipe according to the invention is shown in FIG. 4, which differs from that of FIG. 1 in that the solid fillets 15 are placed helically on the periphery of the pipe with an angle of inclination a relative to the horizontal of between 10° and 89°, preferably between 30° and 85°, and in a more preferred way between 45° and 70°. With reference to FIG. 4, the helical solid fillets are preferably placed in a manner parallel to one another with a uniform spacing between two successive helices. The helical solid fillets generally extend over 10% to 100% of the total height of the pipe. Preferably, the solid fillets 15 extend over a distance of between 50% and 100% of the total height of the pipe. The fillets are thus welded at a certain number of meeting points either with the first face of the vertical wires or with the horizontal support rings.

It is also conceivable to arrange the helical solid fillets so that two adjacent fillets meet at their distal end in a V-shaped configuration.

Alternatively, two adjacent helical fillets can intersect each other in an X shape with a first fillet inclined with an angle α relative to the horizontal and a second fillet inclined with an angle −α relative to the horizontal. In this configuration, the two intersecting fillets are preferably located in the same plane so that the second fillet is not simply “superposed” on the first fillet, but the second fillet is sectioned at each point of crossing with a first fillet and this point undergoes a welding that makes it possible to connect the fillets to each other.

As in the case of the vertical fillets, all of the helical solid fillets can be placed in a zone located in the upper half of the pipe and/or in a zone located in the lower half of the pipe.

Alternatively, the solid fillets cover both a portion of the zones located in the lower and upper halves of the pipe. In an alternative embodiment, the solid fillets are placed successively and alternately in a zone located in the lower half of the pipe and in a zone located in the upper half of the pipe.

In another embodiment, the helical solid fillets 15 are placed so as to form a continuous helix on the periphery of the pipe 8.

A pipe according to the invention can have the following dimensions:
diameter of the pipe: between 0.3 and 2 meters
height of the pipe: between 2 and 30 meters

The number and the dimensions (e.g., the width) of the solid fillets can be determined by knowing the total surface developed by the pipe and by taking into account the fact that the total surface occupied by the solid fillets is between 1 and 30%, preferably between 2 and 20% of the total surfaced developed by the pipe. By way of example, for a pipe of 3.14 m in circumference that comprises four solid fillets extending over the total height of the pipe and occupying 2% of the total surface developed by the pipe, each solid fillet has a width of 1.6 cm.

The applicant has found, surprisingly, that for the same velocity of gas passing through the pipe, the presence of solid fillets that are impermeable to gas on the face of the pipe in contact with the catalyst particles makes it possible to limit the thickness of the particle cake that is jammed by the gas flow relative to a traditional pipe of the prior art made by a network of vertical wires supported by a plurality of horizontal support rings. The presence of these solid fillets thus makes it possible to produce zones where the particles are not so pressed against the wall of the pipe and on which the solid cake cannot grow also with the effect of limiting the thickness of the cake that is formed in the unobstructed zones of the pipe.

Thus, by reducing the amount of catalyst “jammed” by the cake, the portion of “inactive” catalyst is reduced, and therefore the catalytic performance of the reactor is improved.

The invention also relates to a method for catalytic treatment of a hydrocarbon feedstock in a radial reactor having a catalyst moving bed. The reactor comprises:
a casing defining a chamber extending along a vertical main axis and containing a reaction zone having a bed of catalyst particles,
at least one input means of a feedstock,
at least one output means of the effluent produced by the catalytic reaction,
at least one input means of the catalyst for introducing the catalyst into the reaction zone;
at least one output means of the catalyst coming out from the reaction zone; and
optionally, a grid that is permeable to gases and impermeable to catalyst particles, which is placed in the chamber in a manner that is concentric in relation to the collector pipe.

The reactor includes, moreover, a pipe for collecting the effluent according to the invention that is placed in the reaction zone and in communication with the output means of the effluent, it being understood that the solid fillets are in contact with the catalyst particles of the catalytic bed.

A first embodiment of the reactor is shown in FIG. 4, the reactor having centripetal radial circulation (i.e., the gaseous flow circulates from the periphery of the chamber toward the center of the chamber). The reactor comprises a casing 2 that delimits a chamber in which there are arranged a porous cylindrical grid 5 that is permeable to gases and impermeable to the catalyst and a collector pipe 8 according to the invention. The grid 5 is placed between the casing 2 and the collector pipe 8 in a manner that is concentric in relation to the collector pipe. In such a configuration, the reactor comprises an “external” annular zone 20 between the casing 2 and the so-called “external” grid 5, an annular catalytic zone 21 between the so-called “external” grid 5 and the collector pipe 8 and a collector space 22 delimited by the collector pipe 8. As indicated in FIG. 4, the pipe is placed in the reactor so that the solid fillets 15 are immersed in the annular catalytic zone 21 and are thus in contact with the bed of catalyst particles. The so-called “external” grid 5 can take the form of a perforated plate or of a grid formed by a mesh of shaped metal rods and wires or else an assembly of grid elements in the shape of a shell (or “scallop” according to English terminology). During operation, the gaseous feedstock is injected either through the bottom or through the top of the reactor into the annular distribution zone 20, then passes through the so-called “external” grid 5 and next passes in an approximately radial manner through the bed of catalyst particles of the annular catalytic zone 21. In the annular catalytic zone 21, the gaseous fluid is put in contact with the catalyst to produce a reaction effluent, generally gaseous, which is collected in the space 22 of the collector pipe 8 and which is then drawn off either at the top of the reactor (when the feedstock is introduced at the bottom of the reactor) or at the bottom of the reactor (when the feedstock is introduced through the top of the reactor).

According to an alternative embodiment (not shown), the reactor does not have a cylindrical grid 5 but a plurality of distribution tubes, connected to a distribution box (outside or inside the reactor) and immersed in the reaction zone, which make it possible to distribute the gaseous feedstock in the catalytic zone 21.

Another embodiment of the reactor according to the invention having centrifugal radial circulation (i.e., the gaseous flow circulates from the center of the chamber toward the periphery of the chamber) is shown in a cutaway perpendicular to the vertical axis of the reactor in FIG. 5. The collector pipe 8 is arranged between the chamber 2 and the cylindrical grid 5 and in a way that is concentric in relation to the grid 5. In this arrangement, the collector 8 is equivalent to an “external” grid, and the grid 5 corresponds to an “internal” grid. In other words, the reactor comprises an “exterior” collector zone 23 between the casing 2 and the collector pipe 8, an essentially annular catalytic zone 24 delimited by the so-called “internal” grid 5 and the collector pipe 8 and a space for distribution of the gaseous fluid 25 delimited by the cylindrical grid 5. The collector pipe 8 is configured with its solid fillets 15 immersed in the catalytic zone 24 to be in contact with the catalyst particles. During operation, the gaseous feedstock is introduced either through the top or through the bottom of the reactor via the pipe formed by the cylindrical grid 5 (the grid being closed at the end opposite the end in communication with the input means of the feedstock). The feedstock spreads through the grid 5 and passes in an approximately radial manner through the catalytic zone 24 where it is put in contact with the catalyst. A reaction effluent is collected in the space 23 after having spread through the collector pipe 8 and is then drawn off either at the top of the reactor (when the feedstock is introduced at the bottom of the reactor) or at the bottom of the reactor (when the feedstock is introduced through the top of the reactor).

It should be noted that the embodiment of FIG. 5 can be adapted in a variant in which the grid 5 is replaced by a plurality of tubes for distribution of the gaseous fluid, immersed in the reaction zone, and which are connected to a box for distribution of the fluid that can be arranged inside the reactor or outside of the reactor.

It should be noted that the reactor according to the invention can be a reactor having a catalytic moving bed, i.e., the catalyst is introduced into the reactor and drawn off from said reactor continuously.

The reactor and the method according to the invention can be applied to perform reactions having radial circulation of gaseous fluid, such as, for example, a reaction for catalytic reforming of a hydrocarbon fraction, a skeletal isomerization of olefins, the metathesis for the production of propylene, and an oligomeric cracking reaction.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.
In the foregoing and in the examples, all temperatures are set forth uncorrected in degrees Celsius and, all parts and percentages are by weight, unless otherwise indicated.
The entire disclosures of all applications, patents and publications, cited herein and of corresponding application No. FR 1461003, filed Nov. 14, 2014, is incorporated by reference herein.

### Example

A transparent parallepiped mockup 30, using a small-size radial moving bed, was made to study the phenomenon of jamming (“pinning”) of the catalyst particles.

The mockup 30, shown diagrammatically in FIG. 7, comprises a first grid 31 placed at a distance of 80 mm facing a second identical grid 32 that acts as a collector grid. The grids used in the mockup have a height of 120 and 160 mm. The grid 32 is held in the mockup by means of two solid plates 33 and 34 that perform the role of solid fillets described above.

Spherical alumina pellets are put in circulation (1.8 mm in diameter) in a descending movement in the space 35 separating the grids 31 and 32 with a speed of 10 cm/min. An air flow is injected in a current that is approximately perpendicular to the direction of the descending movement of the pellets inside the parallelepiped box so as to reproduce a typical gas-solid interaction of the moving-bed reactor.

By means of the mockup, the change in the shape and size of the cake 36 was studied as a function of the heights of the input and output grids for a gas surface velocity at the input of the first grid 31 of 1.2 m/s.

It was observed that the thickness of the cake has a parabolic shape over the entire height of the second grid 32. Furthermore, it was found that the thickness of the cake is practically zero at the ends of the grids (i.e., at the junction with the solid zones) and maximum at the center of the grid (as shown diagrammatically in FIG. 7).

It was also possible to note that the higher the grid, the greater the thickness of the cake at the center of the grid. Thus, for a gas surface velocity of 1.2 m/s, this thickness is 20 mm for grids of 120 mm in height and 43 mm for the grid of 160 mm.

It can thus be concluded that the maximum thickness of the jammed catalyst cake depends on the corresponding grid surface that is passed through while exiting: the presence of solid zones around this grid has the effect of “breaking” the cake in the area of the grid/solid fillet junction with the effect of reducing the thickness of the catalyst cake formed.

The preceding examples can be repeated with similar success by substituting the generically or specifically described reactants and/or operating conditions of this invention for those used in the preceding examples.
From the foregoing description, one skilled in the art can easily ascertain the essential characteristics of this invention and, without departing from the spirit and scope thereof, can make various changes and modifications of the invention to adapt it to various usages and conditions.

1. Collector pipe (8) that is permeable to a gaseous fluid and impermeable to catalyst particles, with an approximately cylindrical shape extending along an approximately vertical axis having a first surface that can be in contact with the catalyst particles and a second surface opposite the first surface, the pipe comprising a plurality of shaped vertical wires (11) having a first face (12) that can be in contact with the catalyst particles and a second face (13) opposite the first face (12), the shaped wires (11) being integral with a plurality of horizontal support rings (14) by their second face (13), the shaped wires (11) and the support rings (14) defining a plurality of openings, characterized in that the pipe (8) further comprises a plurality of solid fillets (15), impermeable to gaseous fluid and to particles, which are integral with the first face of the shaped wires (11) and/or placed and held between two vertical wires (11), and in that the solid fillets (15) form an angle of inclination a in relation to the horizontal of between 10° and 90°.

2. Collector pipe according to claim 1, wherein the total surface occupied by the solid fillets (15) is between 1 and 30% of the total surface developed by the pipe, preferably between 2 and 20% of the total surface developed by the pipe.

3. Pipe according to claim 1, wherein each solid fillet (15) extends over a height of between 10 and 100% of the total height of the pipe.

4. Collector pipe according to claim 1, wherein the solid fillets (15) are placed in a zone located in the upper half of the pipe and/or in a zone located in the lower half of the pipe.

5. Collector pipe according to claim 1, wherein the solid fillets (15) extend from the upper end or from the lower end of the Pipe.

6. Collector pipe according to claim 1, wherein the solid fillets (15) cover at the same time portions of the zones located in the lower and upper halves of the pipe.

7. Collector pipe according to claim 1, comprising successively and alternately a solid fillet (15) extending in a zone corresponding to the upper half of the pipe and a solid fillet extending in a zone corresponding to the lower half of the pipe.

8. Collector pipe according to claim 1, wherein the solid fillets placed in the upper half of the pipe are arranged facing the solid fillets placed in the lower half of the pipe, and wherein the sum of the heights of the solid fillets facing one another is between 20 and 100% of the total height of the pipe.

9. Collector pipe according to claim 1, wherein the solid fillets are parallel to one another.

10. Collector pipe according to claim 1, wherein the angle of inclination is different from 90°, and wherein two solid fillets (15) meet to form a “V” or intersect to form an “X.”

11. Reactor having radial circulation of gaseous fluid comprising:
an external casing (2) defining a chamber extending along a vertical main axis and containing a reaction zone having a bed of catalyst particles;
at least one input means of a feedstock;
at least one output means of the effluent produced by the catalytic reaction;
at least one input means of the catalyst to introduce the catalyst into the reaction zone;
at least one output means of the catalyst coming out from the reaction zone; and
a pipe (8) for collecting effluent according to one of the preceding claims, in communication with the output means of the effluent, placed in the reaction zone, it being understood that the solid fillets are in contact with the catalyst particles of the catalytic bed, and
optionally, a cylindrical grid (5) that is permeable to gases and impermeable to catalyst particles, placed in the chamber (2) in a manner that is concentric in relation to the collector pipe (8).

12. Reactor according to claim 11, wherein the cylindrical grid (5) is placed between the external casing (2) and the collector pipe (8), wherein the reactor comprises an annular zone for external distribution (20) between the casing (2) and the cylindrical grid (5), an annular catalytic zone (21) between the cylindrical grid (5) and the collector pipe (8), and a collector space (22) delimited by the collector pipe (8), and wherein the first face of the wires of the collector pipe is in contact with the catalytic bed.

13. Reactor according to claim 11, wherein the collector pipe (8) is placed between the casing (2) and the cylindrical grid (5), wherein the reactor comprises an external annular collector zone (23) between the casing (2) and the collector pipe (8), an annular catalytic zone (24) between the cylindrical grid (5) and the collector pipe (8), and a collector space (25) delimited by the cylindrical grid (5), and wherein the first face of the wires of the collector pipe is in contact with the catalytic bed.

14. Method for catalytic conversion of a hydrocarbon feedstock using a reactor according to claim 11, wherein:
the hydrocarbon feedstock is sent continuously in gaseous form into a catalytic bed contained in the reactor;
the hydrocarbon feedstock passing radially through the catalytic bed is put in contact with the catalyst so as to produce a gaseous effluent; and
said effluent is drawn off after it passes through the collector pipe.

15. Method according to claim 13, wherein the catalytic bed is mobile, and the catalyst is continuously sent and drawn off.
